(19) United States
(12) Patent Application Publication
(10) Pub. No.: US 2012/0202971 A1
(43) Pub. Date: Aug. 9, 2012

(54) LIGHT ACTIVATED ASSOCIATION OF SPLIT GFP

(76) Inventors: Kevin P. Kent, Midland, MI (US); Steven G. Boxer, Stanford, CA (US); Keunbong Do, Stanford, CA (US)

(21) Appl. No.: 13/366,821

(22) Filed: Feb. 6, 2012

Related U.S. Application Data
(60) Provisional application No. 61/439,610, filed on Feb. 4, 2011.
(60) Provisional application No. 61/549,865, filed on Oct. 21, 2011.

(51) Int. Cl. C07K 14/00; C12P 21/06

(52) U.S. Cl. 530/350; 435/68.1; 435/69.1

(57) ABSTRACT

The disclosure relates to a split GFP protein. The GFP protein includes a truncated strand and a beta strand (β-strand). The truncated GFP and the synthetic β-strand respond to the presence of light by changing an assembly thereof

### RELATED DOCUMENTS

This patent document claims benefit under 35 U.S.C. §119 to U.S. Provisional Patent Application Ser. No. 61/439,610 entitled “Light Activated Association of Split GFP” and filed on Feb. 4, 2011, and to U.S. Provisional Patent Application Ser. No. 61/549,865 entitled “Beta-Strand Association and Dissociation in Split-GFP System” and filed on Oct. 21, 2011; these patent documents and the Appendices filed in the underlying provisional applications, including the references cited therein, are fully incorporated herein by reference.

### FEDERALLY-SPONSORED RESEARCH AND DEVELOPMENT

This invention was made with Government support under contract GM027738 awarded by the National Institutes of Health. The Government has certain rights in this invention.

### BACKGROUND

Green Fluorescent Protein (GFP) and numerous related fluorescent proteins are used as protein tagging agents. GFP has also been used as a solubility reporter of terminally fused test proteins. GFP is in family of homologous 25-30 kDa polypeptides sharing an 11 beta-strand “barrel” structure. The family currently comprises some 100 members, and includes yellow, red and green fluorescent proteins cloned from various Anthozoa and Hydrozoa species. A wide variety of fluorescent protein label assays and kits are commercially available and encompass a broad spectrum of GFP spectral variants and GFP-like fluorescent proteins.

Reconstitution of split GFP has been described, mainly for detecting protein-protein interactions. For example, fusion proteins connected to the two GFP fragments have been used to drive the joining of the GFP fragments.

### SUMMARY

The present disclosure relates to a split GFP protein, and the association of the two fragments of the GFP protein in response to light, as described herein. The fragments of the GFP can be used in light-activated bioconjugation and to fluorescently label proteins. While the present disclosure is not necessarily limited in these contexts, various aspects of the invention may be appreciated through a discussion of examples using these and other contexts.

Aspects of the present disclosure are directed to a GFP protein split into a truncated strand and a beta strand (β-strand). The truncated GFP strand is refolded after removal of the β-strand. The GFP chromophore in the refolded truncated GFP is in a trans configuration. A synthetic β-strand does not recombine with the truncated GFP strand while its chromophore is in the trans configuration. In the presence of light, the truncated GFP chromophore can change from the trans configuration to a cis configuration or vice versa. In the cis configuration, the truncated GFP will combine with a β-strand present. Rather than light directed assembly, light directed disassembly can occur.

Aspects of the present disclosure are directed to a method of producing a light-activated split GFP. GFP is digested using trypsin, for example, at a loop that isolates strand 10 or 11 from the rest of the protein. The GFP is then denatured to separate a truncated portion of the GFP from strand 10 or 11. The truncated GFP with strand 11 removed refolds into a trans configuration. The refolded truncated GFP does not bind with synthetic β-strand 11. In the presence of light, the truncated GFP is transformed from the trans configuration to a cis configuration. The cis configuration of the truncated GFP binds with any synthetic strand 10 or 11 present in the solution.

Certain aspects of the present disclosure are directed to an assay for detecting, or creating, interaction between two proteins. A truncated GFP in the trans configuration is fused to a first protein. A synthetic β-strand of GFP is fused to a second protein. The truncated GFP and the synthetic β-strand are placed in a solution. The synthetic β-strand and the truncated GFP combine in response to the introduction of light. The amount of first protein and the second protein combined can be detected based on the level of fluorescence given off by the recombined GFP.

Various embodiments, relating to and/or using such methodology and apparatuses, can be appreciated by the skilled artisan, particularly in view of the figures and/or the following discussion.

The above overview is not intended to describe each illustrated embodiment or every implementation of the present disclosure.

### BRIEF DESCRIPTION OF THE DRAWINGS

Various example embodiments may be more completely understood in consideration of the following detailed description in connection with the accompanying drawings, in which:

FIG. 1 shows a method of obtaining split GFP consistent with an embodiment of the present disclosure;

FIG. 2 shows a method of producing light-responsive split GFP, consistent with an embodiment of the present disclosure;

FIG. 3 shows a light-activated split GFP tag, consistent with embodiments of the present disclosure;

FIG. 4 illustrates aspects of a search, consistent with another embodiment of the present disclosure;

FIG. 5 shows a schematic of strand removal and reassembly based on circularly permuted GFP focusing on the 10th beta strand, in accordance with example embodiments of the instant disclosure;

FIG. 6 shows a split GFP complex and split YFP complex, consistent with embodiments of the present disclosure;

FIG. 7A shows the absorbance and fluorescence spectra of s10:loop:GFP and s10203T·s10:loop:GFP, consistent with embodiments of the present disclosure;

FIG. 7B shows the absorbance change of s10:loop:GFP (dark blue) upon addition of s10203T aliquots, consistent with embodiments of the present disclosure;

FIG. 7C shows the absorbance change of s10:loop:GFP upon addition of s10203Y aliquot, consistent with embodiments of the present disclosure;

FIG. 8 shows fluorescence binding titration of 2 nM s10:loop:GFP with s10203Y, consistent with embodiments of the present disclosure;

FIG. 9 shows binding kinetics of 50 nM s10:loop:GFP and 7 μM s10, consistent with embodiments of the present disclosure;

FIG. 10A shows a schematic illustration of a peptide exchange process leading to color change, in accordance with example embodiments of the instant disclosure;

FIG. 10B shows the absorbance change of 1.3 μM s10203T·s10:loop:GFP and 30 μM s10203Y mixture that is kept in the dark and observed over 5 days, consistent with embodiments of the present disclosure;

FIG. 10C shows the absorbance change of 1.3 μM s10203T·s10:loop:GFP and 30 μM s10203Y mixture kept in the dark and observed over 5 days with a 5.7 m·mL-1 of 405 nm light irradiation for 50 minutes, consistent with embodiments of the present disclosure;

FIG. 10D shows a pseudo first-order peptide exchange rate versus the 405 nm laser power per 1 mL sample mixture, consistent with embodiments of the instant disclosure;

FIG. 11A shows a Guanine Nucleotide Exchange Factor (GEF) protein inhibited by split-GFP complementation, consistent with embodiments of the instant disclosure;

FIG. 11B shows the active site of GEF exposed, consistent with embodiments of the instant disclosure, upon split-pair dissociation, which in turn enables activation of small GTPase substrate;

FIG. 11C shows the active form of GEF reversibly trapped by adding strand 10^{203Y}, consistent with example embodiments of the instant disclosure;

FIG. 12 shows an illustration of local cargo delivery guided by light, in accordance with example embodiments of the instant disclosure;

FIG. 13 shows an illustration of a dual-s10 GFP as protease sensor, consistent with embodiments of the instant disclosure; and

FIG. 14 shows a light-activated polymerization of GFP molecules, in accordance with example embodiments of the instant disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure, including aspects defined in the claims.

### DETAILED DESCRIPTION

The present disclosure can be useful for light activated recombination of split GFP. Following light activation of the truncated portion of GFP, the split GFP can force localization of molecules fused to the two split portions of the GFP. The split GFP can also be used to fluorescently label proteins. The β-strand is used to tag the protein, and following light activation of the truncated GFP, the GFP binds with the β-strand. While the present disclosure is not necessarily limited to such applications, various aspects of the disclosure may be appreciated through a discussion of various examples using this context.

In the instant disclosure, the term “synthetic protein” can describe both chemically and biologically synthesized proteins (e.g., synthesized out of a cell or synthesized in the cell). While the present disclosure (which includes the attachments) is amenable to various modifications and alternative forms, specifics thereof have been shown by way of example in the drawings and will be described in further detail. It should be understood that the intention is not to limit the disclosure to the particular embodiments and/or applications described. Various embodiments described above and shown in the figures and attachments may be implemented together and/or in other manners. One or more of the items depicted in the drawings/figures can also be implemented in a more separated or integrated manner, as is useful in accordance with particular applications.

In various embodiments, truncated GFP is a GFP with the eleventh β-strand of the 11 stranded β-barrel or with the tenth β-strand of the 11 stranded β-barrel removed. The truncated GFP, once it has been refolded, does not reassemble with a synthetic peptide when strand 11 is removed, and readily reassembles with a synthetic peptide when strand 10 is removed. However, following light activation, the truncated GFP does reassemble with a synthetic strand 11.

In certain embodiments, split GFPs are useful for making semi-synthetic GFPs containing unnatural amino acids with novel properties for imaging and bioconjucation, and for fundamental studies of β-strand assembly and stability. Split GFPs with one β-strand removed can be obtained for in vitro studies by inserting a peptide loop containing a protease cleavage site between the secondary structural element, to be removed, and the rest of the protein. The loop is cleaved with a protease and the structural element is removed by size exclusion chromatography. Because any of GFP's 11 β-strands or the central helix that contains the chromophore can be made into the C- or N-terminus by circular permutation, this method can be applied to any of the secondary structural elements.

In certain more specific embodiments, a loop between strands 10 and 11 that contains a proteolytic cleavage site is used. This isolates strand 11 from the rest of the protein. The protein is digested with trypsin to make a non-covalent complex GFP. The digest GFP is then denatured in 6M guanidine hydrochloride, for example, to break up the stable non-covalent complex into a truncated GFP fragment and β-strand fragment. Size exclusion chromatography then separates the truncated GFP fragment from the native β-strand fragment while the fragments are in denaturing conditions. When the truncated GFP is diluted out of the denaturant in the presence of a synthetic β-strand, a GFP complex is formed whose absorption and fluorescence spectra are indistinguishable from the original GFP. If, however, the truncated GFP is refolded without synthetic β-strand present, a new species is formed: trans truncated GFP. Surprisingly, trans truncated GFP does not non-covalently associate with the synthetic β-strand. If trans truncated GFP is irradiated, a photostationary state is established between the trans and cis configuration of the chromophore, and cis GFP rapidly combines with the synthetic β-strand to form a partially synthetic GFP. In certain specific embodiments, the properties of the reassembled GFP can be altered by changing the sequence of the synthetic β-strand.

In various embodiments of the present disclosure, a truncated GFP is refolded without a synthetic or native β-strand present. The refolded truncated GFP has an absorption and fluorescence that are different from that of GFP. As used throughout the specification, the cis and trans connotations refer to the chromophores of the truncated GFP in a simple solvent; however, the chromophores may be twisted somewhat from their ideal geometry by constraints in the truncated GFP.

Various embodiments are directed to using the split GFP to bring molecules together at specified times. The split GFP consists of two fragments, a truncated GFP, which includes a chromophore, and a β-strand. In certain more specific embodiments, the β-strand is replaced with a synthetic β-strand. The synthetic β-strand allows for variations in the characteristics of the GFP complex when the two fragments are united. Two molecules of interest are fused to the GFP fragments. A first molecule is fused to the truncated GFP and a second molecule is fused to the synthetic β-strand. When the solution containing the truncated GFP and the synthetic β-strand is exposed to light, the synthetic β-strand and the truncated GFP combine. This brings the two molecules into close proximity and allows for interactions between the two molecules to occur. The combination GFP complex that results can be monitored based on the fluorescence of the GFP complex. Because the truncated GFP and the GFP complex have different fluorescence signatures, it is possible to determine how much of each molecule type has been brought into the presence of the other molecule.

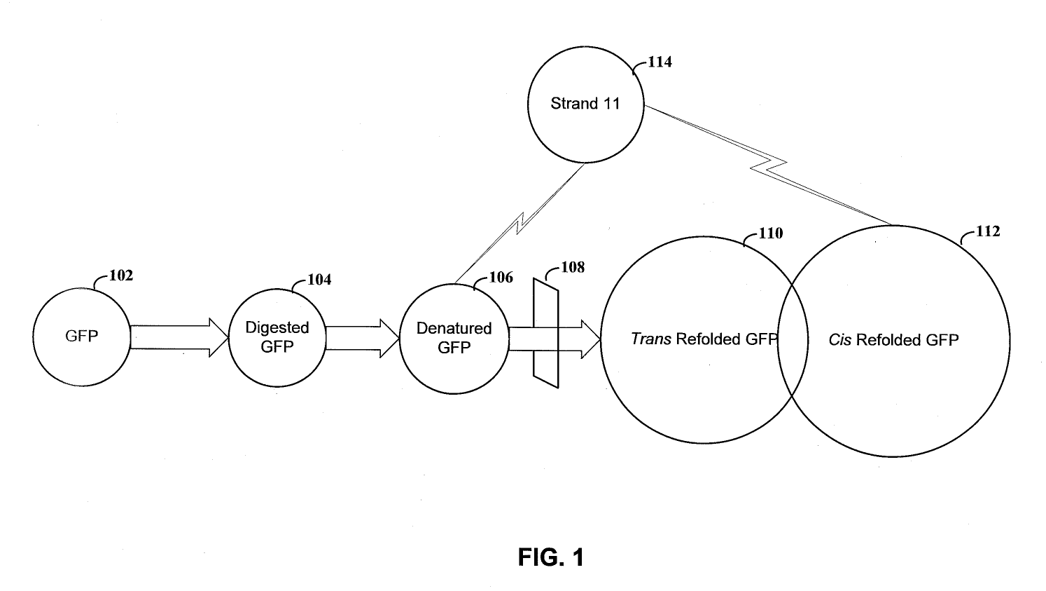

Turning to FIG. 1, a method for producing light-responsive split GFP is depicted, consistent with various embodiments of the present disclosure. GFP 102 is digested using trypsin, for example. The digesting cuts a loop that connects one of the staves of the GFP barrel to the rest of the barrel. After the cut, the digested GFP 104 holds its barrel form, but the strand(s) are no longer connected to the rest of the GFP barrel. The digested GFP 104 is then denatured. The process splits the digested GFP 104 into a denatured truncated GFP 106 and a β-strand. The β-strand is separated from the denatured truncated GFP 106 using a filter 108. In certain specific embodiments, the filter is size exclusion chromatography. The denatured GFP 106 is diluted out of the denaturing solution. In the absence of denaturing solution, the denatured GFP can refold. The truncated GFP that is generated by removing strand 11 and its chromophore refold into trans refolded GFP 110 when the solution is not exposed to light. When exposed to light, the configuration of the chromophore switches to a cis configuration and the GFP becomes cis refolded GFP 110. When synthetic strand 11 is present, the truncated GFP can change between configuration 110 and configuration 112, based on the presence or absence of light. If synthetic β-strand 114 is present after GFP is denatured, but before it is removed from the denaturing solution, the denatured GFP will refold to include the synthetic β-strand 114. If synthetic β-strand is added to the solution after denatured GFP 106 has refolded into trans refolded GFP 110, no interaction occurs between the GFP 110 and the synthetic β-strand 114. However, if light is present and the chromophore changes to the cis configuration, then the synthetic β-strand 114 will combine with the cis refolded GFP 112. The characteristics of the semi-synthetic GFP formed by the combination have similar characteristics to native GFP. However, changes can be made to the β-strand peptide 114. In such instances, the combined GFP can have characteristics that differ from those of native GFP.

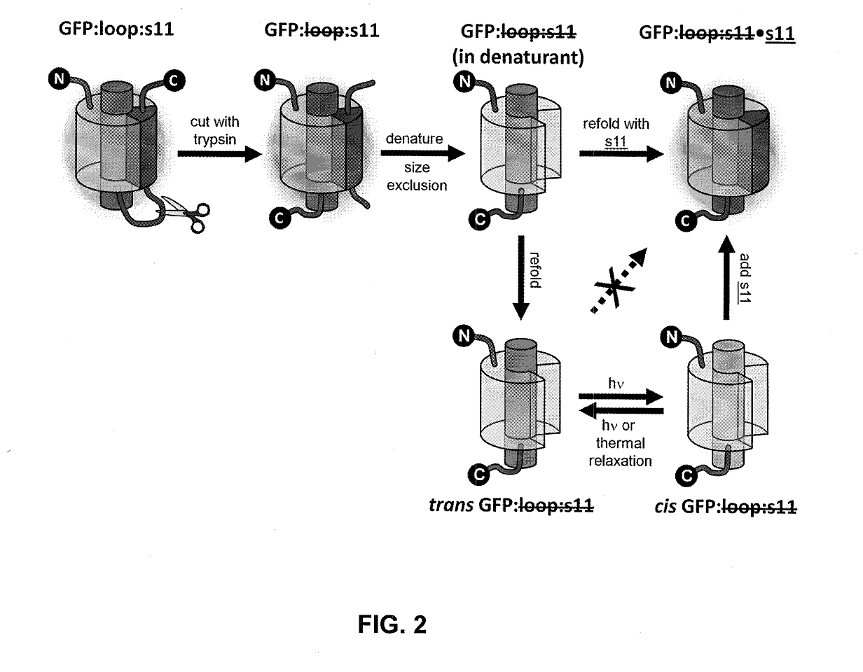

Turning to FIG. 2, a method of producing a light-activated recombination of split GFP is depicted, consistent with embodiments of the present disclosure. GFP:loop:s11 has a loop containing a proteolytic cleavage site that isolates stave 11 from the rest of the protein and is expressed in high yield with the GFP chromophore formed. GFP:loop:s11 has characteristics similar to that of native GFP.GFP:loop:s11 is digested with trypsin to make a non-covalent complex GFP::s11, where the strike-through indicates that the loop is cut, which is then denatured in 6M guanidine hydrochloride to break up the stable noncovalent complex. Size exclusion chromatography then separates GFP: from the native stave 11 in denaturing conditions. When GFP: is diluted out of denaturant in the presence of synthetic strand 11, s11, the GFP:·s11 complex is formed, whose absorption and fluorescence spectra are indistinguishable from the original GFP:loop:s11 (the underline indicates an added synthetic strand). If, however, GFP: is refolded by itself, in the absence of s11, a new species is formed, denoted trans GFP:. Surprisingly, trans GFP: does not non-covalently associate with added s11. If trans GFP: is irradiated, a photostationary state is established between the trans and cis configuration of the chromophore (chromophore trans structure and chromophore cis structure). The cis GFP: rapidly combines with s11 to form GFP::s11, whose properties are indistinguishable from the original GFP:loop:s11.

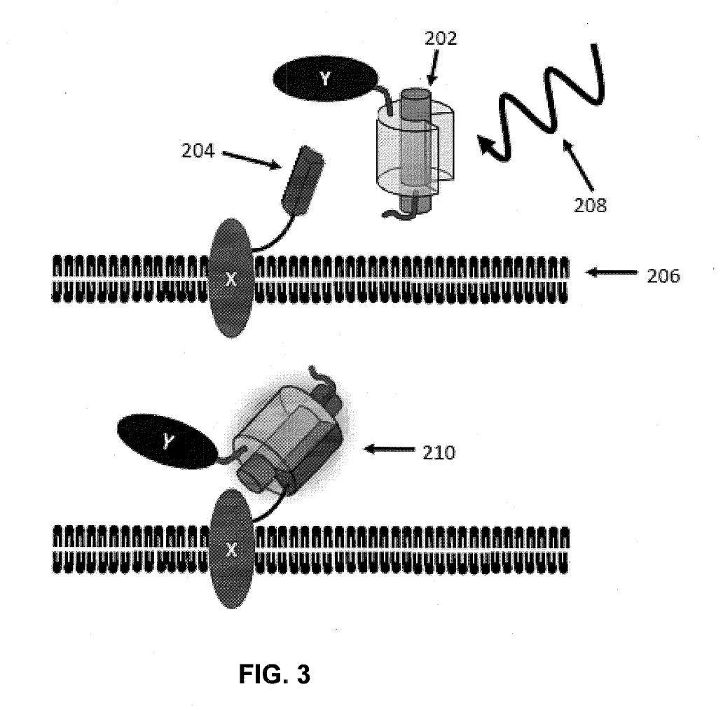

Turning to FIG. 3, a fragment of split GFP is used as a tag, consistent with various embodiments of the present disclosure. A β-strand 204 is attached to a membrane 206 either as an extension of a membrane protein or in a synthetic membrane-anchored form. When truncated GFP 202 is present, light 208 causes β-strand 204 and truncated GFP 202 to combine, resulting in GFP complex 210, which fluoresces. This forces the compound X to interact with the compound Y because they are tethered to 204 and 202, respectively.

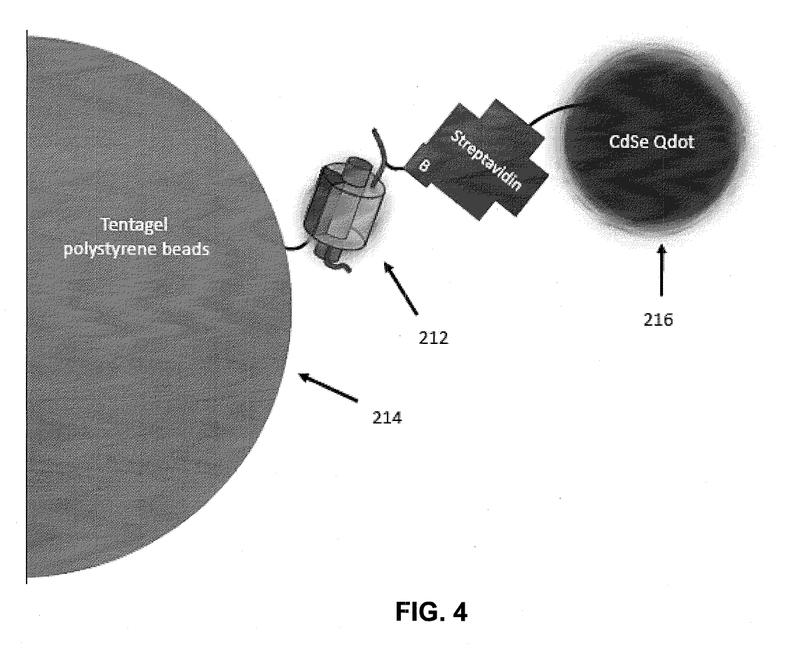

Beta strands that impart new properties to the split GFP complex, such as light-activated dissociation of the GFP::s11 complex, may be discovered by searching peptide libraries according to the following method, which is consistent with another aspect of the present disclosure. With reference to FIG. 4, libraries of peptides are synthesized by mix and split solid phase synthesis. These peptide libraries are incubated with biotin labeled truncated GFP to make the GFP::s11 complex (212) while the peptide library is still attached to the solid phase synthesis beads (214). Following complex formation, the beads are incubated with streptavidin-coated quantum dots (216) and the peptides that interact with the split GFP are selected by the quantum dot fluorescence. Lastly, MALDI MS/MS is used to determine the peptide sequence. New properties are selected for amongst the positive hits either when the complex is attached to the bead, or in solution after determining the sequence of positive hits and synthesizing the peptides. In another embodiment, a similar approach is undertaken using phage display or any method of making large libraries of peptides.

The chromophore changed and strand 11 would not rebind unless light was shined to activate the reassembly. In certain applications and experiments, this feature can be used to target elements associated with strand 11 to wherever the truncated (strand 11-less) protein is located.

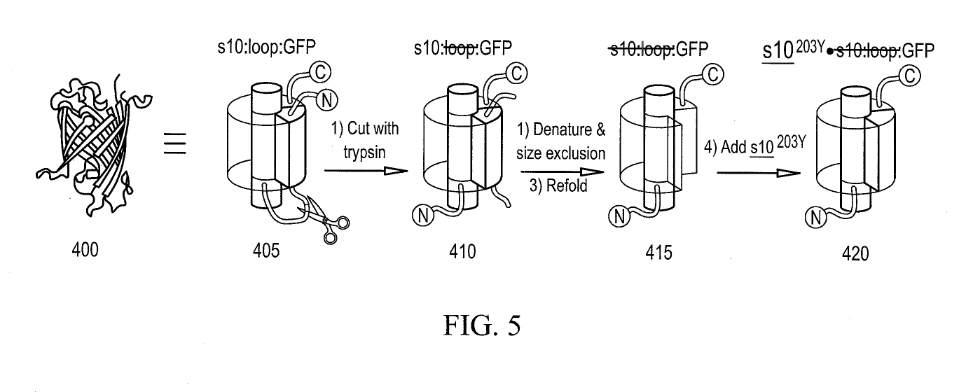

Split green fluorescent proteins (GFPs), along with other split reporter proteins, have been developed as probes to study protein-protein interactions and protein localization in cells. The spontaneous reassembly of split proteins can also be used to generate semi-synthetic proteins in vitro, in which the smaller fragment can be prepared with complete synthetic control. The method and notation illustrated in FIG. 5 can be generally applied to any secondary structural element of GFP, e.g., all 11 beta strands and the central helix containing the chromophore. A circularly permuted GFP is expressed with a protease cleavage site inserted in a loop. The cleavage site is added between the secondary structural element, to be removed, and the rest of the protein. The cleavage site is cut, and the secondary structural element is removed by size exclusion chromatography in denaturing conditions to obtain the truncated protein. Interestingly, when the truncated GFP with the 11th strand removed (GFP:loop:s11) is refolded, the chromophore undergoes thermal cis-to-trans isomerization. Strand 11 does not bind to the trans truncated GFP, but binds only to the cis truncated GFP after making a photostationary mixture of cis and trans truncated GFPs. Kinetic and thermodynamic studies of the reassembly process are complicated due to this unique binding. By contrast, a truncated GFP refolded with the 10th strand removed (s10:loop:GFP), as shown in FIG. 5, binds to synthetic strand 10 without such complications, permitting direct and quantitative measurement of the reassembly process. Furthermore, strand 10 contains threonine 203 that causes a red-shift upon mutation to tyrosine (T203Y), which is the basis of the widely-used class of yellow fluorescent proteins (YFPs), and which provides a convenient way of probing strand replacement as illustrated in FIG. 5.

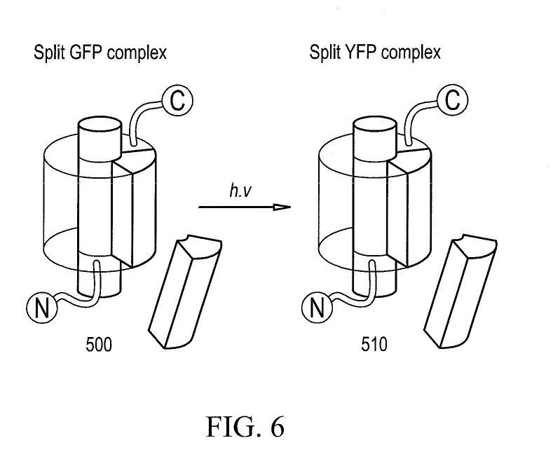

Turning now to FIG. 5, which shows a schematic of strand removal and reassembly based on circularly permuted GFP focusing on the 10th beta strand. At 400, a cartoon tertiary structure of a GFP is shown adapted from a PDB structure of superfold GFP (2B3P). Circularly permuted GFP with strand 10 at its N-terminus is connected to the rest of the protein through a loop sequence containing a protease cleavage site, and is denoted as s10:loop:GFP 405 (the ordering of elements is always from N- to C-terminus). A strike through loop, s10::GFP 410, indicates the protease cleavage site was cut, and an additional strike through s10, GFP 415, indicates that the native strand 10 was removed and that the truncated protein is refolded. An underlined s10, s10, refers to an added synthetic strand 10 that forms a complex with the truncated GFP. In FIG. 4, the T203Y mutation is shown at 420, which changes the color of the reassembled protein as in YFP. FIG. 6 shows a split GFP complex 500 and a split YFP complex 510. The split GFP complex 500 shifts to the split YFP complex 510 under the influence of light. The GFP in the diagram is shown as a cylinder with a strand simply removed, however, this is an interpretation of the structure for visualization purposes. Similarly, although the beta strands are presented as wedges, their secondary structure is likely to change after binding to the truncated GFP.

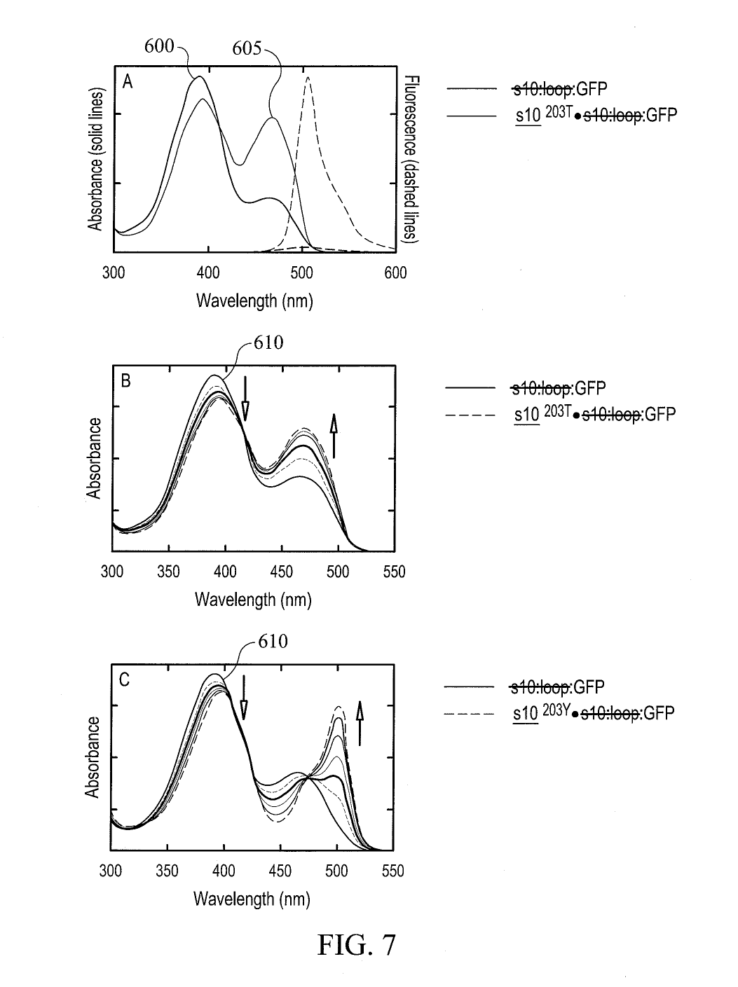

FIG. 7 shows reconstitution of GFP from s10 and :GFP. FIG. 7A shows a comparison of the absorbance and the fluorescence emission spectra before and after the complex formation between s10^{203T} and GFP. FIG. 7A specifically shows the absorbance and fluorescence spectra of :GFP (600) and s10^{203T}·s10:loop:GFP (605). All spectra in FIG. 7 are normalized by concentration so that relative absorbance and fluorescence intensity directly translate to the relative extinction coefficient and the product of extinction coefficient and fluorescence quantum yield. Both the absorbance and the fluorescence spectra becomes nearly indistinguishable from those of the un-cut protein (s10:loop:GFP) once the complex is formed. Upon complex formation, both protonated and deprotonated absorbance bands, respectively at 389 nm and 465 nm, are slightly red-shifted to 393 nm and 467 nm, with an isosbestic point around 410 nm. The truncated protein is only weakly fluorescent, and the fluorescence quantum yield shows a large increase (about 30-fold for 390 nm excitation and 505 nm emission) when the peptide binds. The spectral shift and dramatic increase in fluorescence quantum efficiency are useful for the acquisition of kinetic and thermodynamic data of the reassembly process. This information can be further exploited in imaging applications. Weak fluorescence is reminiscent of what is observed for the isolated chromophore. This indicates that removal of strand 10 results in conformational flexibility that leads to non-radiative decay. By comparison, when strand 11 is removed, the absorbance spectrum changes substantially as the trans form of the chromophore is formed, and fluorescence is reduced by a factor of 36.

FIGS. 7B and 7C show the absorbance change of :GFP when it is titrated with s10^{203T} or s10^{203Y} to reform GFP or YFP, respectively. More specifically, FIG. 7B shows the absorbance change of :GFP (610) upon addition of s10^{203T} aliquots. FIG. 7C shows the absorbance change of GFP upon addition of s10^{203Y} aliquots. In FIGS. 7B and 7C arrows indicate the direction of spectral changes as more peptide is added, and the dotted curves are the spectra of purified GFP or YFP complex, normalized at the isosbestic points, showing the expected final spectra upon reconstitution.

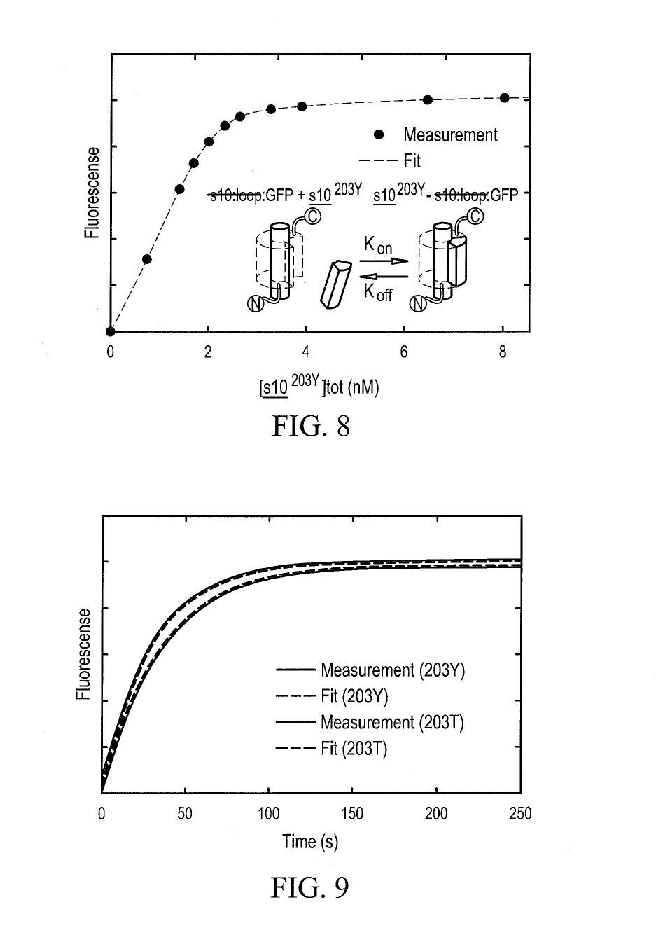

The equilibrium constant of the binding reaction was measured using fluorescence quantum yield recovery as an indication for the complex formation. FIG. 8 shows a plot of the fluorescence intensity as a function of the total concentration of s10^{203Y} mixed with 2 nM :GFP. The sample in FIG. 8 was excited at 500 nm, and emission was collected at 520 nm. Each data point is an average of 4 different sample measurements, and error bars indicate standard deviation. The data were fit to the analytical solution of a one-to-one binding reaction, giving a dissociation constant (K_{d}) of 78.7+13.8 pM. In a similar manner, K_{d}=139.1±20.1 pM was determined for s10^{203T}. These K_{d} values are much smaller than the value reported for strand 7 complementation (531 nM), and even smaller than the lowest value for fragment complementation (1.5 nM in the presence of 750 mM glycerol), which is one of the highest affinities for protein-protein interactions involving β-strands.

The K_{d} values were too small to be precisely measured by isothermal calorimetry given the small heat generated per binding reaction. However, the standard enthalpy of reaction) (ΔH°) can be obtained by measuring the total heat released from a single injection of s10 (4.3 M excess) into 1.4 mL of 500 nM :GFP. The resulting ΔH° can be used with the equilibrium constant to obtain entropy ΔS° (all values of which are summarized in Table 1).

Surprisingly, an apparent enthalpy-entropy compensation for T203Y substitution leads to a relatively small difference in the free energy of binding (ΔΔG°=ΔG°_{203Y}−ΔG°_{203T}=−0.34±0.13 kcal·mol⁻¹) despite the large difference in ΔH° (ΔΔH°=ΔH°_{203Y}−ΔH°_{203T}=−10.38±2.36 kcal·mol⁻¹). Because the only difference between the two systems is the T203Y substitution, this provides an estimate of the energetic consequences of a single side-chain difference.

TABLE 1
Thermodynamic and kinetic parameters of s10•:GFP interaction at 25° C., 1 atm.

| s10 | K_{d}^{a} | ΔG [symbol] ^{b} | ΔH [symbol] | ΔS [symbol] | kₒₙ | k_{off} | K_{d} [symbol] |
|---|---|---|---|---|---|---|---|
| peptide | (pM) | (kcal · mol⁻¹) | (kcal · mol⁻¹) | (cal · mol² · K¹) | (M⁻¹s⁻¹) | (s⁻¹) | (pM) |
| s10^{203T} | 139.1 ± 20.1 | −13.43 ± 0.09 | −26.29 ± 1.46 | −43.16 ± 4.90 | 4232 ± 163 | 6.08 × 10⁻⁷ ± 6.57 × 10⁻⁸ | 143.8 ± 16.5 |
| s10^{203Y} | 78.7 ± 13.8 | −13.77 ± 0.10 | −36.67 ± 1.86 | −76.86 ± 6.25 | 5658 ± 135 | 3.43 × 10⁻⁷ ± 2.07 × 10⁻⁷ | 60.65 ± 36.64 |

^{a}From direct titration.
^{b}Calculated from K_{d}^{a} [symbol]
From k_{off}/kₒₙ ratio. [symbol]
indicates data missing or illegible when filed

As shown in FIG. 9, the association (on-) rate of s10 and :GFP was measured using fluorescence recovery with great care not to expose the sample to any more light than needed for the reasons discussed below. FIG. 9 shows binding kinetics of 50 nM :GFP and 7 μM s10. Emission at 505 nm for s10^{203T} reassembly and 520 nm for s10^{203Y} reassembly was monitored while exciting the protein at 390 nm. Kinetic fits were performed by numerically solving the differential equations of a bimolecular reaction. From the fits, bimolecular rate constants of 4232±163 M⁻¹s⁻¹ and 5658±135 M⁻¹s⁻¹ were determined respectively for s10^{203T} and s10^{203Y} binding (Table 1). These association rates are about 30 fold faster than that reported for strand 11 association to the cis form of GFP:.

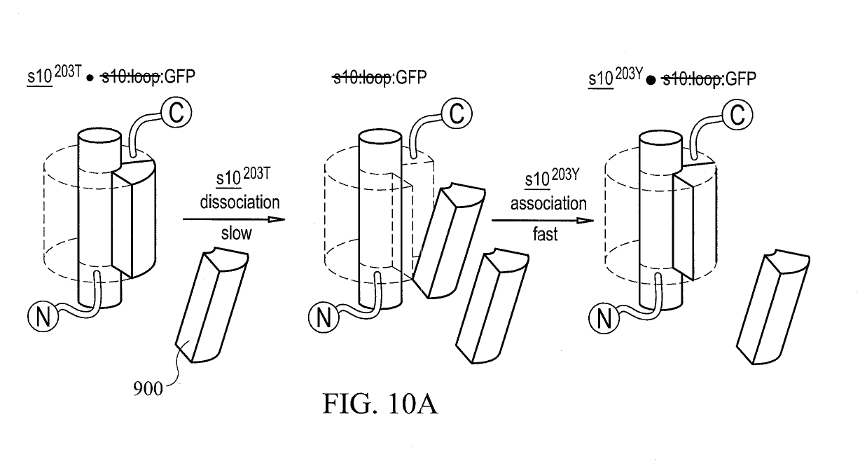

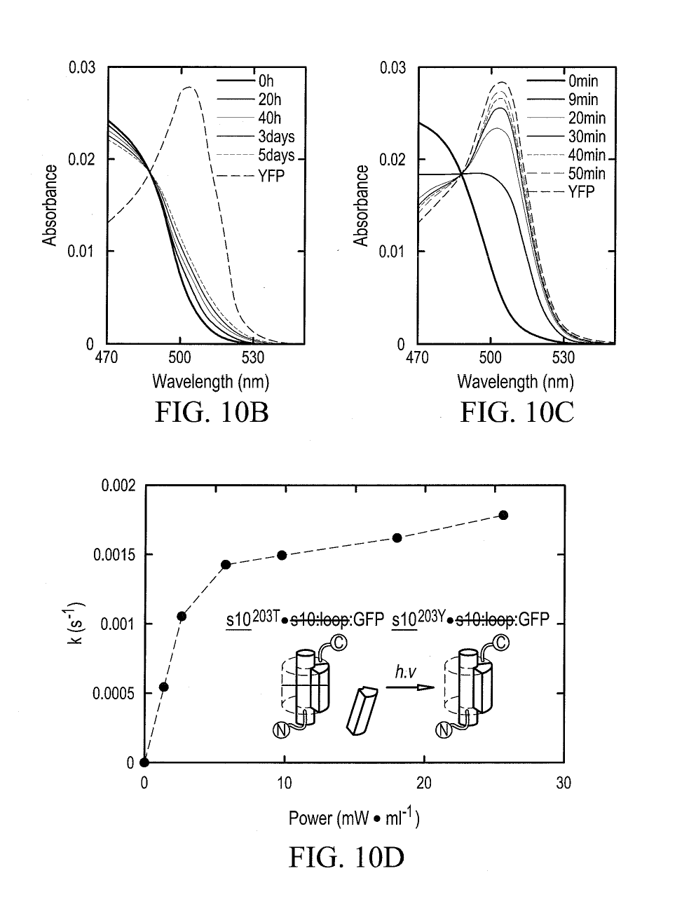

Turning now to FIG. 10A, which shows schematic illustration of the peptide exchange process leading to color change (the wedge 900 represents the excess s10^{203Y}). FIG. 10B shows the absorbance change of 1.3 μM s10^{203T}:GFP and 30 μM s10^{203Y} mixture in the dark observed over 5 days (t½≈300 hours) and FIG. 10C shows an absorbance spectrum change with 5.7 mW·mL⁻¹ of 405 nm light irradiation for 50 minutes (t_{1/2}=8 minutes). FIG. 10D shows pseudo first-order peptide exchange rate versus the 405 nm laser power per 1 mL sample mixture. FIGS. 10A-10D are further discussed below.

When the GFP complex (s10^{203T}·:GFP) was mixed with excess s10^{203Y}, the absorbance shifted very slowly from that of GFP to that of YFP as shown in FIG. 10B. The spectral shift occurs in the other direction, from the YFP to the GFP spectrum, when the YFP complex (s10^{203Y}·:GFP) was mixed with excess s10^{203T}. This indicates that a non-covalently bound strand can be spontaneously replaced by an added strand without denaturing the protein. The exchange process can be described with a simple two-step model as schematically illustrated in FIG. 10A: first, the native strand dissociates, and second, the different strand binds to the truncated protein.

Taking advantage of the spectral shift accompanying the peptide exchange, the dissociation (off-) rates of the complexes could be estimated by adding the different peptide in excess. For example, as shown in FIG. 10B, gradual conversion of GFP to YFP was observed with a half-life of about 300 hours. Since the half-life of the YFP complex formation process (:GFP+s10^{203Y}→s10^{203Y}·:GFP) would be only 4 s in 30 μM s10^{203Y}, the dissociation step of the exchange process must be rate-limiting, and thus the dissociation rate can be estimated directly from the exchange rate (as shown in Table 1). Taking the ratio of the dissociation and the association rates, K_{d} values of 143.8±16.5 pM for s10^{203T} and 60.65±36.64 pM for s10^{203Y} were obtained, which agree with the K_{d} values obtained from the binding isotherm within their error. Thus, the peptide exchange process appears to be well described by the scheme shown in FIG. 10A.

Surprisingly, the peptide exchange rate was dramatically enhanced by light irradiation. As shown by comparing FIGS. 10B and 10C, the apparent exchange rate was up to 3000 times greater in the presence of light, suggesting that the rate-limiting step of the exchange process, the dissociation of s10^{203T} in this case, is effectively accelerated by light. FIG. 10D is a plot of the peptide exchange rate as a function of the power of a 405 nm continuous wave diode laser irradiating a 1 mL mixture of 1.3 μM s10^{203T}·:GFP and 30 μM s10^{203Y} that is constantly stirred. The rate increases linearly in the lower power range and levels off at higher power. The quantum yield of the peptide exchange process was approximately 0.2% in the linear region (up to about 4 mW·mL⁻¹).

When either of the complexes, s10^{203T}·:GFP or s10^{203Y}·:GFP, was exposed to 405 nm light without adding extra peptide in solution, the absorbance spectrum shifted toward that of :GFP and the fluorescence intensity decreased accordingly (as can be seen in FIG. 7A). Assuming that the peptide photodissociates from the truncated protein to give a mixture of the complex and the dissociated species, the equilibrium composition in the presence of light could be properly predicted with the measured association rates (Table 1) and the light-enhanced dissociation rates. Once the irradiation was stopped, absorbance and fluorescence returned to those of the starting complex over time. Furthermore, when a bimolecular reaction model was numerically fit to the absorbance and fluorescence recovery data, rate constants of 4205±576 M⁻¹s⁻¹ and 5606±303 M⁻¹s⁻¹ were determined respectively for the GFP and the YFP complex, which is within the error of the independently measured association rate of each peptide (as seen in Table 1). This agreement shows that the light irradiation is indeed facilitating the peptide to dissociate.

Similar to :s11 which binds to strand 11 only with the cis configuration of the chromophore, it is possible that the chromophore in s10·:GFP is in the cis configuration, and rapidly undergoes reversible cis-to-trans isomerization upon photoexcitation, where the putative trans s10·s10:loop:GFP has an enhanced dissociation rate for strand 10. Such light-driven dissociation of a GFP peptide can be an effective way of introducing perturbations to a biological system with high spatial and temporal resolution. Furthermore, spectral shifts caused by mutations such as T203Y allow reversible and orthogonal enhancement of s10^{203T} and s10^{203Y} dissociation.

The split-GFP scheme, with its built-in fluorescent reporter, provides a reliable and convenient platform to extract kinetic and thermodynamic information of a split system, with access to complete synthetic flexibility on a given strand.

Light-induced dissociation of strand 10 can be used as a light-driven switch to turn on and off various protein activities. Although there have been efforts to obtain spatial and temporal control of protein-protein interactions, these methods tend to use toxic ultra-violet irradiation, require cofactors and/or are not readily reversible, so the development of a caged system that is genetically encoded could see many applications.

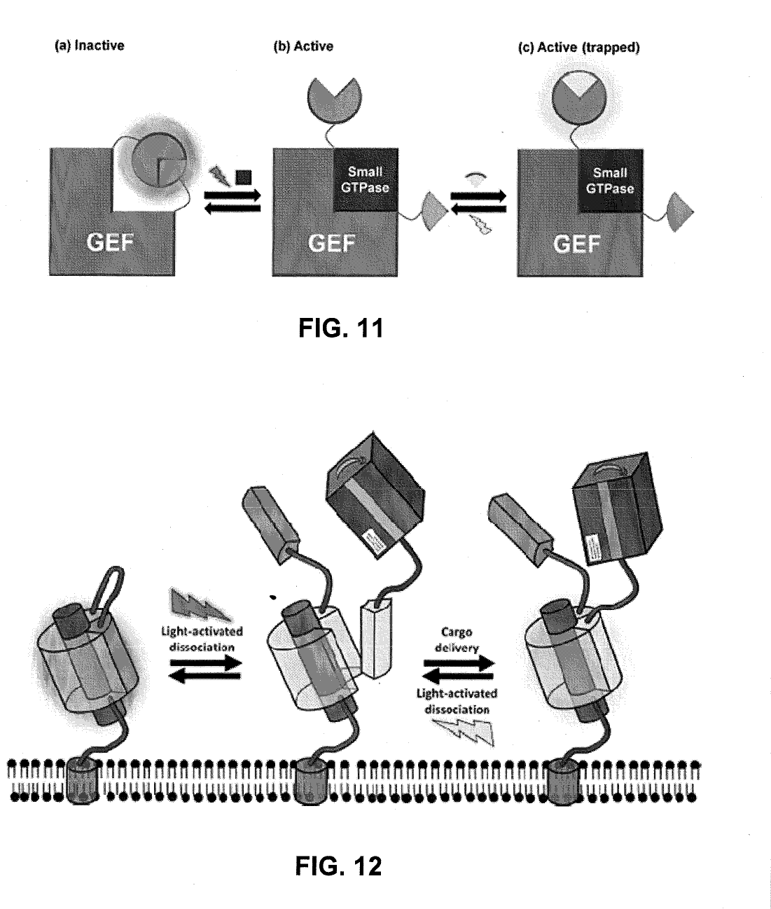

FIG. 11 is an illustration of a usage of split-GFP as a reversible photoswitch of an enzyme activity. In the figure, a Guanine Nucleotide Exchange Factor (GEF) is initially inhibited sterically by split-GFP complementation, as is shown in FIG. 11A. Upon illumination that facilitates dissociation of the split pair, GEF can bind to its substrate, small GTPase, and become enzymatically active, as shown in FIG. 11B. When the light is no longer present, the split pair will reassemble to turn the enzymatic activity off. An activated form of the enzyme can also be “trapped” by adding or co-expressing strand 10203Y (foaming YFP). (Strand 10203Y can be expressed at the terminus close to the GFP side of the chimeric enzyme). This has two advantages: 1) the degree of activation can be monitored via ratiometric fluorescence detection and 2) the degree of enzyme activation can be pushed to completion (without strand 10203Y the degree of activation is limited by the photostationary state). It should be noted that this can be done without sacrificing reversibility of the switch since the active form, shown in FIG. 11C, can be illuminated with light absorbed by the YFP to return to the inactive foam.

In certain embodiments, the scheme is also expanded to obtain orthogonal control over two different signaling pathways. For instance, when strand 10203T and strand 10203Y are used respectively to form split GFP and YFP pairs for Intersectin (ITSN) and Tiam inhibition, their respective substrates, Cdc42 and Rac, can be selectively activated by illuminating the cell with the appropriate wavelength. This will be observed as a light-induced increase in filopodia or lamellipodia. Such control is useful to manipulate multiple protein pathways and understand cross-talk between multiple signaling pathways in living cell.

Another way of regulating small-GTPase-mediated signaling processes is to translocate small-GTPase to the plasma membrane. This is accomplished by expressing GFP anchored to the membrane facing the cytoplasm, as illustrated in FIG. 12. The molecule to be recruited to the plasma membrane, such as Cdc42 or Rac, is fused to strand 10203Y, and upon light activation of the GFP, the cargo is delivered to the targeted location by heterodimerization, thus completing the translocation. The heterodimerization is detected by ratiometric fluorescence change, and is reverted by exciting with wavelength absorbed by YFP. When the protein or the peptide is expressed on a cell surface, biologically important cargoes, such as signaling molecules, are delivered to cells (or to a small area of a single cell) that are selectively exposed to light. Such spatial control is useful to study inter-cellular signaling or cell migration. Again, the success of the cargo delivery is monitored by ratiometric spectral shift, and the aforementioned peptides with unnatural amino acids used as an ex vivo reagent for cargo delivery.

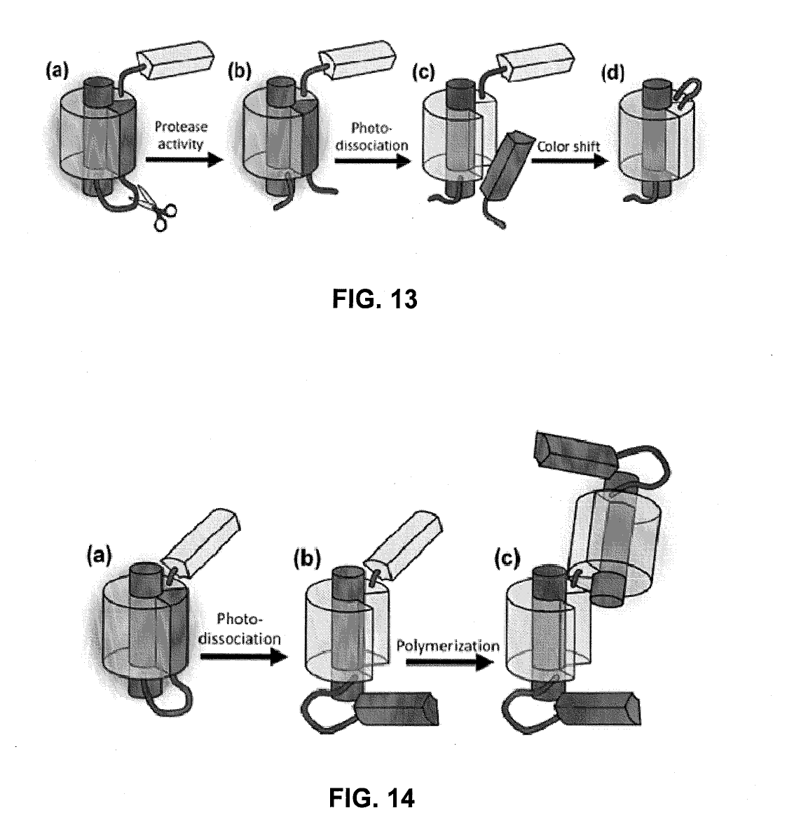

A GFP with strand 10 on both the N- and the C-terminus (“dual-s10 GFP”) is used as a sensor. Certain protease activity correlates well with cancer, where such quantitative sensing is valuable. GFPs are resistant to protease activity. A floppy loop sequence with an arginine residue in the middle was engineered as a protease cut site (no other arginines were cut). More generally, when a certain recognition site (e.g., a receptor domain) is engineered into the loop, and when binding of a target molecule induces dissociation of the adjacent strand 10 (i.e., the native strand 10), the other strand will replace the native strand and give a color shift. The advantage of the scheme is to sense molecules and protein activity ratiometrically. FIG. 13 illustrates the usage of a GFP as a protease sensor, in accordance with the instance disclosure. FIG. 13A, shows the protease cleavage site is inserted in the loop that connects the natively bound strand 10. With the protease activity, the loop is cut, which is shown in FIG. 13B, and thus the native strand is photodissociated, as seen in FIG. 13C. Finally, as shown in FIG. 13D, the other strand replaces the native strand to generate ratiometric color shift.

Turning now to FIG. 14, light activated polymerization of GFP molecules is shown. When the linker of one strand 10 (YFP strand in this case) is made short enough to prevent it from binding to itself (i.e., the protein to which the strand is covalently bound), photodissociation of the native strand (GFP strand in this case) results in polymerization of GFP molecules. The YFP strand of the second GFP is not shown in (c) to avoid congestion in FIG. 14.

Strand 10 can be brought to the end of the protein by circularly permuting the sequence. In such instances, when strand 10 is removed, the chromophore does not change, and therefore, strand 10 rebinding can be measured directly (kinetics and thermodynamics become possible). Light enhances the exchange rate of peptide strands (when one strand replaces another, the color changes). Further, light effectively dissociates strand 10 from the protein.

Strand exchange can be used as a mechanism for light-directed modulation of pathways in cells. GFP, with strand 10 cut, can be considered a “caged protein” (e.g., a protein that changes its properties in response to light).

Aspects of the instant disclosure are directed towards a split green fluorescent protein (GFP). According to a general embodiment, the GFP includes a refolded truncated GFP (e.g. strands 1-10 of a GFP), and a synthetic β-strand. The refolded truncated GFP and the synthetic β-strand are configured and arranged for responding to the presence of light by changing an assembly thereof. In certain more specific embodiments, the assembly change includes a light-directed assembly of the refolded truncated GFP and the synthetic β-strand which is a synthetic version of the eleventh strand of a green fluorescent protein. In other more embodiments, the assembly change includes a light-directed disassembly of the refolded truncated GFP and the synthetic β-strand which is a synthetic version of the tenth strand of a green fluorescent protein. Split GFP complexes, in accordance with the instant disclosure, can be utilized to tag a protein with the synthetic β-strand. This can label the protein for further experimentation and observation. Often times, the refolded truncated GFP and synthetic β-strand, described above, are located within a cell or can be located in vivo. Each of the complexes can additionally include another synthetic β-strand if desired.

The split GFP complex can be utilized for observation of a protein-protein interaction of interest. In those instances, the refolded truncated GFP is attached to a first protein, and the synthetic β-strand is attached to a second protein.

Aspects of the instant disclosure are additionally directed towards methods that involve green fluorescent proteins. For instance, in an example embodiment of methods consistent with the instant disclosure, a green florescent protein (GFP) is digested, in a solution, that isolates a stave from the rest of the protein. Certain specific embodiments utilize strand 11 as the stave. The GFP is then denatured to break up a truncated portion of the GFP and the stave, and the stave is removed from the solution. The truncated protein of the GFP is refolded, and a truncated GFP chromophore in a trans configuration results. Through light activation, a synthetic stave is introduced and combined with the truncated GFP in the solution. Embodiments utilizing strand 11 as the stave also utilize synthetic strand 11 for the synthetic stave. Certain embodiments of methods consistent with the instant disclosure can additionally utilize at least one of the synthetic stave and the truncated GFP to tag a protein.

Methods, consistent with the instant disclosure, can also be further described in that providing light to the truncated GFP in trans configuration transitions the truncated GFP to a cis configuration. Methods can further include introducing at least one of the trans truncated GFP and the synthetic stave into cell. Additionally, methods can include tagging a protein of interest with the synthetic stave, or, in other instances, a peptide can be tagged with the trans truncated GFP. Additionally, methods of the instant disclosure can be utilized to label a first protein of interest and a second protein of interest. In those instances, the first protein is labeled with a trans truncated GFP, and the second protein is labeled with a synthetic stave. This allows for observation of the protein-protein interactions.

The instant disclosure is also directed towards further methods involving GFP proteins. These methods involve placing a certain secondary structural element (a beta strand in certain instances), or any element of a green fluorescent protein (GFP), at the N-terminus or the C-terminus of the protein connected to a loop sequence that contains a protease cleavage site. The GFP is digested at the loop in a solution, and an element is isolated from the rest of the protein. The methods further including denaturing the GFP to break up a truncated portion of the GFP and the element of interest, and removing the element from the solution. The truncated portion of the GFP is refolded, being in a trans configuration. A synthetic left-out element is combined to the truncated GFP, through light activation by introducing the synthetic left-out element to the solution with the truncated GFP. Certain specific embodiments are further characterized in that the element is strand 11 of the GFP, and the synthetic left-out element is a synthetic strand 11.

In certain embodiments, methods utilizing the synthetic left-out element can include at least one of the synthetic left-out element and the truncated GFP to tag a protein. Further, providing light to the truncated GFP in trans configuration transitions the truncated GFP to a cis configuration. Additionally, methods can include introducing at least one of the trans truncated GFP and the left-out element into a cell. Proteins of interested can be tagged with the synthetic left-out element in order to observe, for example, protein kinetics and interaction. A peptide can also be tagged with the trans truncated GFP. In order to observe interactions of two proteins, methods can further include labeling a first protein with the trans truncated GFP and a second protein with the left-out element.

Aspects of the instant disclosure are also directed towards methods that force the interaction of two proteins (X and Y). In such methods, a truncated GFP in the trans configuration fused to a protein X is presented in a solution. Also in that solution, a β-strand of GFP, fused to protein Y, is presented. The strand of GFP combines with the truncated GFP in response to light. The fluorescence level of the solution is then detected. The β-strand of GFP is fused to a membrane protein, in methods that force interaction of two proteins. Further, in methods that force two proteins to interact, the fluorescent spectrum of the solution shifts after the introduction of light to the solution. Certain aspects of methods that force the interaction of two proteins, consistent with the instant disclosure, can convert the truncated GFP from a trans configuration to a cis configuration in the presence of activating light. Additionally, these methods can be further described in that the truncated GFP in the trans configuration does not combine with the β-strand unless light is added. The truncated GFP includes strands 1-10, utilized in methods that force interaction of two proteins, and the β-strand is strand 11. In those instances, the truncated GFP also includes strands 1-10. The truncated GFP, utilized in methods of the instant disclosure, can be attached to a membrane protein. Further, in instances where methods include a truncated GFP, the presence of deactivating light can convert the truncated GFP from a cis configuration to a trans configuration.

Aspects of the instant disclosure are also directed towards assays for determining het peptide sequence of new beta strands that bind to cis truncated GFP. Assays will involve making a random library of potential β-strands, and mixing them with biotin labeled truncated GFP in the presence of light. The potential split GFP complexes are labeled with streptavidin coated quantum dots. The β-strands that interact with cis truncated GFP are selected by looking at the quantum dot fluorescence, and determining the peptide sequence by cleaving the peptide off of the beads and determining the sequence by MALDI MS/MS. In certain instances, assays will not include light activation. Therefore, the truncated GFP will be left in the trans configuration. Further, beads selected for quantum dot fluorescence, in assays consistent with the instant disclosure, can be incubated and subjected to competition from a solution phase GFP 10 β-strand to select for displaceable β-strands. Alternatively, beads selected for quantum dot fluorescence, in assays consistent with the instant disclosure, can be incubated and subjected to competition from a solution phase GFP 10 β-strand in the presence of light to select for light-activated displaceable β-strands. Similarly, a solution phase GFP 11 β-strand can be used rather than a solution phase GFP 10 β-strand.

The instant disclosure additionally includes methods of protein activity regulation. Such methods include inhibiting a guanine nucleotide exchange factor (GEF) by split-GFP complementation. An enzimatically active site of the GEF is exposed by illuminating the split-GFP complementation to facilitate dissociation of the split-GFP complementation and to facilitate binding of the GEF to small GTPase; and reversibly trapping the enzymatically active GEF by adding strand 10^{203Y} either exogenously or endogenously. Methods of protein activity can additionally involve expressing GFP anchored to a membrane. The GFP is facing the cytoplasm of a cell (or the extracellular matrix). A Cdc42 or Rac molecule is fused to strand 10^{203Y}, and the GFP is activated by illumination. Next, the Cdc42 or Rac molecule and the strand 10^{203Y} are translocated to the GFP by heterodimerization.

Additionally, the instant disclosure is directed towards a protease sensor, which includes a GFP including strand 10 on both an N-terminus and C-terminus of the GFP. The GFP itself includes a floppy loop with a protease cleavage site (e.g., arginine residue for trypsin) or a molecular binding site inserted into the GFP. Through this formation, the GFP is designed to sense molecules or protein activity ratiometrically in response to a target molecule binding to a receptor cite in the floppy loop. This binding induces dissociation of a native strand 10, and replaces the native strand with a different strand 10 thereby producing a color shift or in response to a covalent bond breakage caused by a protease activity.

Molecule-based apparatuses and methods of protein regulation are designed based on the instant disclosure. For example, such molecule-based apparatuses and methods of protein regulation are directed towards activation and deactivation of protein function with light (e.g., by spatially blocking and opening up the active site with light). Additionally, molecule-based apparatuses and methods of protein regulation can involve inserting a sensing domain (e.g., a protease cleavage site, binding site) in the loop that connects to the native, thermodynamically more stable, strand. In the instances where a sensing domain is inserted in the loop that connects to the native strand, the sensing is initiated with light, in the presence of the target molecule or the activity, in which the native strand does not bind back; and the replacing strand shifts the color (ratiometric sensing). Moreover, the molecule-based apparatuses and methods of protein regulation can be designed for cargo delivery to a target area of a protein by locally facilitating peptide exchange, or for light-activated protein polymerization.

Various embodiments described above and shown in the figures may be implemented together and/or in other manners. One or more of the items depicted in the drawings/figures can also be implemented in a more separated or integrated manner, or removed and/or rendered as inoperable in certain cases, as is useful in accordance with particular applications. Embodiments involving the creation of split GFP may be used in connection with various tagging and bioconjugation. Further, various forms of synthetic β-strand can be used to differentiate multiple molecules tagged at one time. In view of the description herein, those skilled in the art will recognize that many changes may be made thereto without departing from the spirit and scope of the present invention.

Based upon the above discussion and illustrations, those skilled in the art will readily recognize that various modifications and changes may be made without strictly following the exemplary embodiments and applications illustrated and described herein. Furthermore, various features of the different embodiments may be implemented in various combinations. Such modifications do not depart from the true spirit and scope of the present disclosure, including those set forth in the following claims.

What is claimed is:

1. A split green fluorescent protein (GFP) complex comprising:
a refolded truncated GFP and
a synthetic β-strand, the refolded truncated GFP and the synthetic β-strand configured and arranged for responding to the presence of light by changing an assembly thereof.

2. The split GFP complex of claim 1, wherein the assembly change includes a light-directed assembly of the refolded truncated GFP and the synthetic β-strand which is a synthetic version of the eleventh strand of a green fluorescent protein.

3. The split GFP complex of claim 1, wherein the assembly change includes a light-directed disassembly of the refolded truncated GFP and the synthetic β-strand which is a synthetic version of the tenth strand of a green fluorescent protein.

4. The split GFP complex of claim 1, wherein a protein is tagged with the synthetic β-strand.

5. The split GFP complex of claim 1, wherein the refolded truncated GFP is attached to a first protein involved in a protein-protein interaction of interest, and the synthetic β-strand is attached to a second protein involved in the protein-protein interaction of interest.

6. A method comprising:
digesting, in a solution, a green fluorescent protein (GFP) at a loop that isolates a stave from the rest of the protein;
denaturing the GFP to break up a truncated portion of the GFP and the stave, and removing the stave from the solution;
refolding the truncated portion of the GFP, resulting in the truncated GFP being in a trans configuration; and
combining, through light activation, a synthetic stave introduced to the solution and the truncated GFP.

7. The method of claim 6, wherein the stave is strand 11, and the synthetic stave is synthetic strand 11.

8. The method of claim 6, wherein at least one of the synthetic stave and the truncated GFP are used to tag a protein.

9. The method of claim 6, wherein providing light to the truncated GFP in trans configuration transitions the truncated GFP to a cis configuration.

10. The method of claim 6, further including introducing at least one of the trans truncated GFP and the synthetic stave into or onto a cell.

11. The method of claim 6, further including tagging a protein of interest with the synthetic stave.

12. The method of claim 6, further including tagging a peptide with the trans truncated GFP.

13. The method of claim 6, further including labeling a first protein with the trans truncated GFP and a second protein with the synthetic stave.

14. A method comprising:
placing a certain secondary structural element or any element of a green fluorescent protein (GFP) at the N-terminus or the C-terminus of the protein connected to a loop sequence that contains a protease cleavage site;
digesting the GFP at the loop, in a solution, isolating an element from the rest of the protein;
denaturing the GFP to break up a truncated portion of the GFP and the element, and removing the element from the solution;
refolding the truncated portion of the GFP, resulting in the truncated GFP being in a trans configuration; and
combining, through light activation, a synthetic left-out element introduced to the solution and the truncated GFP.

15. The method of claim 14, wherein the secondary structural element is a beta strand.

16. The method of claim 14, wherein the element is strand 11 of the GFP, and the synthetic left-out element is a synthetic strand 11.

17. The method of claim 14, further including labeling a first protein with the trans truncated GFP and a second protein with the left-out element.

18. The method of claim 14, wherein at least one of the synthetic left-out element and the truncated GFP are used to tag a protein.
